(19) United States
(12) Patent Application Publication
(10) Pub. No.: US 2025/0135140 A1
(43) Pub. Date: May 1, 2025

(54) FACIAL RECOGNITION-BASED BREATHING MASK MATCHING METHOD AND SYSTEM THEREOF

(71) Applicant: RESVENT MEDICAL TECHNOLOGY CO., LTD, SHENZHEN (CN)

(72) Inventors: YUTONG WANG, SHENZHEN (CN); WEI WANG, SHENZHEN (CN)

(21) Appl. No.: 18/493,816

(22) Filed: Oct. 25, 2023

(30) Foreign Application Priority Data
Dec. 21, 2022 (CN) 202211651550.0

(51) Int. Cl. A61M 16/06; A61M 16/00

(52) U.S. Cl. CPC A61M 16/0605; A61M 16/024

(57) ABSTRACT

A facial recognition-based breathing mask matching method and a facial recognition-based breathing mask matching system are disclosed. The facial recognition-based breathing mask matching method includes determining a breathing mask type suitable for a user according to basic information of the user, collecting facial information and head information of the user according to the breathing mask type suitable for the user, establishing a breathing mask model according to collected facial information and collected head information of the user, and matching a modeling result with breathing masks in a breathing mask database to select the most suitable breathing mask for the user. By adopting the facial recognition-based breathing mask matching method, the most suitable breathing mask is selected for the user.

### TECHNICAL FIELD

The present disclosure relates to a field of breathing masks, and in particular to a facial recognition-based breathing mask matching method and a facial recognition-based breathing mask matching system.

### BACKGROUND

Currently, there are many types of breathing masks on the market, which are usually divided into three types: oral and nasal masks, nasal masks, and nasal pillows. According to different sizes of faces, the breathing masks are further divided into three types: small size, medium size, and large size, and according to different functions, the breathing masks have different shapes.

When the breathing masks of the prior art are matched with faces of users, there are following technical problems:
1. When existing companies provide the breathing masks to the users, the users are unable to experience and try out the breathing masks;
2. Repeated try-on of the breathing masks increases a risk of infection; and
3. The users have poor compliance when performing home positive pressure ventilation therapy, which is generally caused by wearing inappropriate breathing masks.

### SUMMARY

A purpose of the present disclosure is to propose a facial recognition-based breathing mask matching method and a facial recognition-based breathing mask matching system to solve a technical problem of inconvenient fitting between a conventional breathing mask and a face of a user in the prior art.

Embodiments of the present disclosure provides the facial recognition-based breathing mask matching method. The facial recognition-based breathing mask matching method comprises:
determining a breathing mask type suitable for a user according to basic information of the user;
collecting facial information and head information of the user according to the breathing mask type suitable for the user;
establishing a breathing mask model according to collected facial information and collected head information of the user; and
matching a modeling result with breathing masks in a breathing mask database to select the most suitable breathing mask for the user.

In the embodiments of the present disclosure, the basic information of the user comprises gender of the user, race of the user, whether the use has sensitive skin, whether the use has a sleep breathing problem, whether the use has a beard, whether the user has a wound on a face, and whether the user has a wound on a nose.

In the embodiments of the present disclosure, the basic information of the gender of the user, the race of the user, whether the use has the beard, whether the user has the wound on the face, and whether the user has the wound on the nose is obtained through facial scanning, and the basic information of whether the user has the sensitive skin and the sleep breathing problems is obtained through manual input.

In the embodiments of the present disclosure, the facial recognition-based breathing mask matching method further comprises matching the modeling result with breathing mask components in the breathing mask database, selecting most matching breathing mask components, and then assembling the most matching breathing mask components into a complete breathing mask and recommending the complete breathing mask to the user.

In the embodiments of the present disclosure, the facial recognition-based breathing mask matching method further comprises comparing the modeling result with a current breathing mask worn by the user to evaluate a fit degree of the current breathing mask worn by the user.

The embodiments of the present disclosure further provide the facial recognition-based breathing mask matching system. The facial recognition-based breathing mask matching system comprises a breathing mask type determining module, a breathing mask information collection module, a breathing mask modeling module, a breathing mask database, and a breathing mask matching module. The breathing mask type determining module is configured to determine a breathing mask type suitable for a user according to basic information of the user. The breathing mask information collection module is configured to collect facial information and head information of the user according to the breathing mask type suitable for the user. The breathing mask modeling module is configured to establish a breathing mask model according to collected facial information and collected head information of the user. The breathing mask database is configured to store parameter data of different types of breathing masks and parameter data of breathing mask components forming the breathing masks. The breathing mask matching module is configured to match a modeling result with the breathing masks in the breathing mask database to select the most suitable breathing mask for the user.

In the embodiments of the present disclosure, the basic information of the user comprises gender of the user, race of the user, whether the use has sensitive skin, whether the use has a sleep breathing problem, whether the use has a beard, whether the user has a wound on a face, and whether the user has a wound on a nose.

In the embodiments of the present disclosure, the basic information of the gender of the user, the race of the user, whether the use has the beard, whether the user has the wound on the face, and whether the user has the wound on the nose is obtained through facial scanning, and the basic information of whether the user has the sensitive skin and the sleep breathing problems is obtained through manual input.

In the embodiments of the present disclosure, the facial recognition-based breathing mask matching system further comprises a breathing mask component matching module configured to match the modeling result with the breathing mask components in the breathing mask database and select most matching breathing mask components. The breathing mask component matching module is further configured to assemble the most matching breathing mask components into a complete breathing mask and recommend the complete breathing mask to the user.

In the embodiments of the present disclosure, the facial recognition-based breathing mask matching system further comprises a fit degree evaluation module configured to match the modeling result with a current breathing mask worn by the user and evaluate a fit degree of the current breathing mask worn by the user.

Compared with the prior art, in the facial recognition-based breathing mask matching method and the facial recognition-based breathing mask matching system, the breathing mask type suitable for the user is determined according to the basic information of the user, the facial information and the head information of the user are collected according to the breathing mask type suitable for the user, the breathing mask model is established according to the collected facial information and the collected head information of the user, and the modeling result is matched with the breathing masks in the breathing mask database to select the most suitable breathing mask for the user. Therefore, the most suitable breathing mask is selected for the user without trying the breathing masks on by the user, which reduces costs of the user's offline try-on, breaks limitations of time and space, and reduces a risk of cross-infection. Further, it is also allowed to select the most matching breathing mask components according to the modeling result, and the most matching breathing mask components are assembled into the complete breathing mask recommended to the user, so as to customize a personalized breathing mask for the user, which further improves adaptability of the breathing mask.

### BRIEF DESCRIPTION OF DRAWINGS

FIG. 1 is a flow chart of a facial recognition-based breathing mask matching method according to one embodiment of the present disclosure.

FIG. 2 is a schematic diagram of a facial recognition-based breathing mask matching system according to one embodiment of the present disclosure.

### DETAILED DESCRIPTION

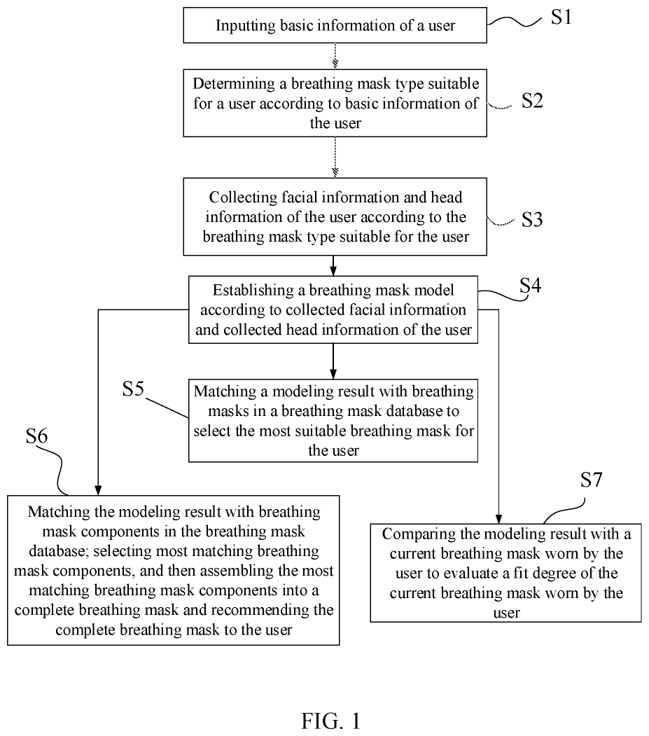

As shown in FIG. 1, embodiments of the present disclosure provide a facial recognition-based breathing mask matching method. The facial recognition-based breathing mask matching method comprises steps S1-S7, which are separately illustrated as follow.
Step S1: inputting basic information of a user.

The basic information of the user comprises gender of the user, race of the user, whether the use has sensitive skin, whether the use has a sleep breathing problem, whether the use has a beard, whether the user has a wound on a face, and whether the user has a wound on a nose. In the embodiments of the present disclosure, the basic information of the user is input into an application ((APP) terminal of a mobile phone, and the basic information of the user is input by the user at the APP terminal and/or by facial scanning of the APP terminal. In the embodiments of the present disclosure, the basic information of the gender of the user, the race of the user, whether the use has the beard, whether the user has the wound on the face, and whether the user has the wound on the nose is obtained through facial scanning, and the basic information of whether the user has the sensitive skin and the sleep breathing problems is obtained through manual input.
Step S2: determining a breathing mask type suitable for a user according to basic information of the user;

It should be noted that the basic information of the user is the key for matching a breathing mask, e.g., if the user has the sensitive skin, skin-friendly memory sponge needs to be used, if the user has a wound on the mouth, a nose mask and a nasal pillow need to be used, if the user has the wound on the nose, the nasal pillow needs to be used, and if the user has the beard on the face, the nasal pillow needs to be used. Further, different types of breathing masks need to be adopted according to different types of face shapes of races of different users.
Step S3: collecting facial information and head information of the user according to the breathing mask type suitable for the user;

It should be noted that parts of the face that need to be collected by different types of breathing masks are also different. For example, for a mouth-nose mask, a main frame thereof is determined by positioning points, such as a nose root position, a left corner of the mouth, a right corner of the mouth, and a one-cm point below the mouth. For a nasal mask, key positioning information comprises a height, a width, and a tip trend of the nose. For the nasal pillow, a shape of nostrils and a width of a nose wing are considered. For a headband, the head information needs to be collected to obtain key skeleton point information.

In the embodiments of the present disclosure, the user is prompted to use a camera of the mobile phone to align with corresponding parts of the face to collect the facial information and the head information in the APP terminal.
Step S4: establishing a breathing mask model according to collected facial information and collected head information of the user;

It should be noted that after the facial information and the head information are collected, modeling is carried out in combination with key skeleton point information of the face and head to obtain a three-dimensional structural model of the breathing mask (i.e., the breathing mask model).
Step S5: matching a modeling result with breathing masks in a breathing mask database to select the most suitable breathing mask for the user;

It should be noted that the breathing mask database stores parameter data of different types of breathing masks and parameter data of breathing mask components forming the breathing masks.

The parameter data of the breathing masks mainly comprises three-dimensional structure data and material data thereof. Three-dimensional structure data of the breathing mask model according to the face information and the head information of the user is compared with the three-dimensional structure data of each of the breathing masks in the breathing mask database, and a breathing mask having the parameter data closest to the three-dimensional structure data of the breathing mask model is selected as the most suitable breathing mask for the user. In this way, the most suitable breathing mask is selected for the user without trying the breathing masks on by the user, which reduces costs of the user's offline try-on, breaks limitations of time and space, and reduces a risk of cross-infection.
Step S6: matching the modeling result with breathing mask components in the breathing mask database, selecting most matching breathing mask components, and then assembling the most matching breathing mask components into a complete breathing mask and recommending the complete breathing mask to the user; and

It should be noted that in order to achieve personalized customization of the breathing mask for the user, the three-dimensional structure data of the breathing mask model according to the face information and the head information of the user is matched with each of the breathing mask components, and the most matching breathing mask components are assembled into the complete breathing mask, so that the complete breathing mask obtained through assembly is more adaptive to the face and the head of the user compared with a conventional breathing mask. Further, the breathing mask is customized for the user, which realize customization of the breathing masks and further improves the adaptability of the breathing masks.
Step S7: comparing the modeling result with a current breathing mask worn by the user to evaluate a fit degree of the current breathing mask worn by the user.

It should be noted that, in some cases, the user needs to evaluate the fitting degree between the current breathing mask worn by the user and the face and the head of the user, and the three-dimensional structure data of the breathing mask model according to the face information and the head information of the user is compared with structural data of the current breathing mask worn by the user, so that the fitting degree of the current breathing mask worn by the user is obtained. Specifically, a total error of the current breathing mask worn by the user is calculated according to an error of each piece of the structural data, and then the fit degree is evaluated according to the total error.

It should also be noted that the above steps S5-S7 are configured to realize different functions based on the modeling result of the breathing mask model, that is, the Steps S5-S7 are independent steps and are not steps having specific orders.

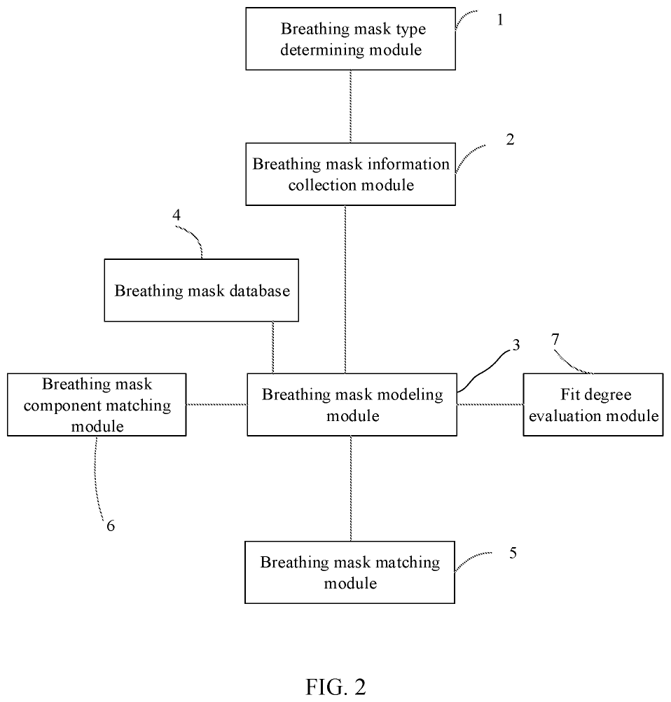

As shown in FIG. 2, corresponding to the facial recognition-based breathing mask matching method, the embodiments of the present disclosure further provide a facial recognition-based breathing mask matching system. The facial recognition-based breathing mask matching system comprises a breathing mask type determining module 1, a breathing mask information collection module 2, a breathing mask modeling module 3, a breathing mask database 4, a breathing mask matching module 5, a breathing mask component matching module 6, and a fit degree evaluation module 7.

The breathing mask type determining module 1 is configured to determine the breathing mask type suitable for the user according to the basic information of the user. The basic information of the user comprises the gender of the user, the race of the user, whether the use has the sensitive skin, whether the use has the sleep breathing problem, whether the use has the beard, whether the user has the wound on the face, and whether the user has the wound on the nose. In the embodiments of the present disclosure, the basic information of the gender of the user, the race of the user, whether the use has the beard, whether the user has the wound on the face, and whether the user has the wound on the nose is obtained through facial scanning, and the basic information of whether the user has the sensitive skin and the sleep breathing problems is obtained through manual input.

The breathing mask information collection module 2 is configured to collect the facial information and the head information of the user according to the breathing mask type suitable for the user. It should be noted that the breathing mask information collection module 2 is a module in the APP terminal of the mobile phone that controls the camera.

The breathing mask modeling module 3 is configured to establish the breathing mask model according to the collected facial information and the collected head information of the user.

The breathing mask database 4 is configured to store the parameter data of different types of the breathing masks and the parameter data of the breathing mask components forming the breathing masks.

The breathing mask matching module 5 is configured to match the modeling result with the breathing masks in the breathing mask database to select the most suitable breathing mask for the user.

The breathing mask component matching module 6 is configured to match the modeling result with the breathing mask components in the breathing mask database and select the most matching breathing mask components. The breathing mask component matching module 6 is further configured to assemble the most matching breathing mask components into the complete breathing mask and recommend the complete breathing mask to the user.

The fit degree evaluation module 7 is configured to match the modeling result with the current breathing mask worn by the user and evaluate the fit degree of the current breathing mask worn by the user.

It should be noted that, in the facial recognition-based breathing mask matching system, content such as information interaction and execution processes between modules/units thereof is same as that of the facial recognition-based breathing mask matching method mentioned above, an implementation process and technical effects brought by the facial recognition-based breathing mask matching system are same as that of the facial recognition-based breathing mask matching methods, and specific content thereof may refer to related descriptions in the facial recognition-based breathing mask matching method, and details are not described therein.

To sum up, in the facial recognition-based breathing mask matching method and the facial recognition-based breathing mask matching system, the breathing mask type suitable for the user is determined according to the basic information of the user, the facial information and the head information of the user are collected according to the breathing mask type suitable for the user, the breathing mask model is established according to the collected facial information and the collected head information of the user, and the modeling result is matched with the breathing masks in the breathing mask database to select the most suitable breathing mask for the user. Therefore, the most suitable breathing mask is selected for the user without trying the breathing masks on by the user, which reduces the costs of the user's offline try-on, breaks the limitations of time and space, and reduces the risk of cross-infection. Further, it is also allowed to select the most matching breathing mask components according to the modeling result, and the most matching breathing mask components are assembled into the complete breathing mask recommended to the user, so as to customize the personalized breathing mask for the user, which further improves the adaptability of the breathing mask.

Foregoing descriptions are only optional embodiments of the present disclosure and are not intended to limit the present disclosure. Any modification, equivalent replacement, or improvement within the technical scope of the present disclosure should be included in the protection scope of the present disclosure.

What is claimed is:

1. A facial recognition-based breathing mask matching method, comprising:
determining a breathing mask type suitable for a user according to basic information of the user;
collecting facial information and head information of the user according to the breathing mask type suitable for the user;
establishing a breathing mask model according to collected facial information and collected head information of the user; and
matching a modeling result with breathing masks in a breathing mask database to select the most suitable breathing mask for the user.

2. The facial recognition-based breathing mask matching method according to claim 1, wherein the basic information of the user comprises gender of the user, race of the user, whether the use has sensitive skin, whether the use has a sleep breathing problem, whether the use has a beard, whether the user has a wound on a face, and whether the user has a wound on a nose.

3. The facial recognition-based breathing mask matching method according to claim 2, wherein the basic information of the gender of the user, the race of the user, whether the use has the beard, whether the user has the wound on the face, and whether the user has the wound on the nose is obtained through facial scanning, and the basic information of whether the user has the sensitive skin and the sleep breathing problems is obtained through manual input.

4. The facial recognition-based breathing mask matching method according to claim 1, wherein the facial recognition-based breathing mask matching method further comprises matching the modeling result with breathing mask components in the breathing mask database; selecting most matching breathing mask components, and then assembling the most matching breathing mask components into a complete breathing mask and recommending the complete breathing mask to the user.

5. The facial recognition-based breathing mask matching method according to claim 1, wherein the facial recognition-based breathing mask matching method further comprises comparing the modeling result with a current breathing mask worn by the user to evaluate a fit degree of the current breathing mask worn by the user.

6. A facial recognition-based breathing mask matching system, comprising:
a breathing mask type determining module configured to determine a breathing mask type suitable for a user according to basic information of the user;
a breathing mask information collection module configured to collect facial information and head information of the user according to the breathing mask type suitable for the user;
a breathing mask modeling module configured to establish a breathing mask model according to collected facial information and collected head information of the user;
a breathing mask database configured to store parameter data of different types of breathing masks and parameter data of breathing mask components forming the breathing masks; and
a breathing mask matching module configured to match a modeling result with the breathing masks in the breathing mask database to select the most suitable breathing mask for the user.

7. The facial recognition-based breathing mask matching system according to claim 6, wherein the basic information of the user comprises gender of the user, race of the user, whether the use has sensitive skin, whether the use has a sleep breathing problem, whether the use has a beard, whether the user has a wound on a face, and whether the user has a wound on a nose.

8. The facial recognition-based breathing mask matching system according to claim 7, wherein the basic information of the gender of the user, the race of the user, whether the use has the beard, whether the user has the wound on the face, and whether the user has the wound on the nose is obtained through facial scanning, and the basic information of whether the user has the sensitive skin and the sleep breathing problems is obtained through manual input.

9. The facial recognition-based breathing mask matching system according to claim 6, wherein the facial recognition-based breathing mask matching system further comprises a breathing mask component matching module configured to match the modeling result with the breathing mask components in the breathing mask database and select most matching breathing mask components; the breathing mask component matching module is further configured to assemble the most matching breathing mask components into a complete breathing mask and recommend the complete breathing mask to the user.

10. The facial recognition-based breathing mask matching system according to claim 6, wherein the facial recognition-based breathing mask matching system further comprises a fit degree evaluation module configured to match the modeling result with a current breathing mask worn by the user and evaluate a fit degree of the current breathing mask worn by the user.
